(19) United States
(12) Patent Application Publication
(10) Pub. No.: US 2013/0330164 A1
(43) Pub. Date: Dec. 12, 2013

(54) IV Pole Transportation Device and Method

(71) Applicant: Applied Ingenuity. LLC, Westbrook, ME (US)

(72) Inventors: MARK T BUSHEY, WINDHAM, ME (US); EDGAR W VETRONE, HOLLIS, ME (US); JOHN P MCLAUGHLIN, Standish, ME (US)

(21) Appl. No.: 13/914,623

(22) Filed: Jun. 10, 2013

Related U.S. Application Data
(60) Provisional application No. 61/658,310, filed on Jun. 11, 2012.

(51) Int. Cl. A61M 5/14

(52) U.S. Cl. CPC A61M 5/1415
USPC 414/800; 280/411.1

(57) ABSTRACT

The present invention is directed to an IV pole transportation device that enables a user to securely engage multiple IV poles into an integrated unit, allowing the IV poles to be transported simultaneously. The IV pole transportation device comprises a base plate having a plurality of slots and a plurality of ratchet assemblies. The ratchet assemblies are secured to the base plate and positioned such that the each ratchet assembly slot is aligned with a base plate slot.

### CROSS-REFERENCE TO RELATED APPLICATION

This application claims the benefit of U.S. Provisional Patent Application No. 61/658,310, filed Jun. 11, 2012, the disclosure of which is herein incorporated by reference.

### BACKGROUND

1. Field of the Invention

The present invention pertains to the field of transportation devices. Specifically, this invention relates to a novel device and method of use for transporting multiple IV poles simultaneously.

2. Discussion of Background Information

IV poles are commonplace in medical and patient care environments and are commonly used to support an IV bag, which may contain blood, medicine or a saline solution. As the name implies, most IV poles utilize a pole or rod to suspend the IV bag above the patient. In addition, IV poles are often used to support pumps or other specialized medical equipment. While IV poles may be fixedly attached to patient support structures, such as beds, stretchers and wheelchairs, individual, mobile IV poles are ubiquitous in most health care settings. One common form of IV pole incorporates a wheeled base, which provides stability and allows patients and medical staff to easily move the IV pole from one location to another.

Hospitals and other health care environments frequently need to move a group of IV poles from one location to another. The most common method to complete this task requires human labor, where transport personnel grasp one or two IV poles and wheel them from one location to another. Moving more than two IV poles simultaneously is difficult and fraught with danger. Most mobile IV poles utilize a wheeled base with either four or five legs. While these configurations provide a stable base while moving individual IV poles, the positioning of the legs proves problematic for transporting multiple IV poles simultaneously.

Specifically, the wheeled legs make it difficult to bring the IV poles close enough together where they can be grasped in one hand. Instead, a person looking to transport multiple IV poles must tip the poles slightly, which significantly decreases the stability of the IV poles by raising one or more wheeled legs off the ground and increases the risk that the IV poles will fall over and damage any attached equipment. Even if the transporter is able to grasp more than one IV pole in his or her hand, maneuvering the poles is difficult and the likelihood of damaging the equipment attached to the IV pole, either because the IV poles banged into an object during transport or because one or more IV poles fell over, is greatly increased.

Various devices have been proposed to address the problem of attaching IV poles to support structures, such as hospital beds, stretchers, wheelchairs, or even walkers. In addition, multiple devices have been proposed to address the problems of attaching equipment to IV poles. However, none of these inventions, taken either singly or in combination, adequately address or resolve the aforementioned problems. Therefore, a need exists for an IV pole transportation device and method that facilitates the simultaneous transport of multiple IV poles.

### SUMMARY OF THE INVENTION

The present invention solves the problems associated with simultaneously transporting multiple IV poles and provides a device and method for securing multiple IV poles together during transport.

The present invention is directed to an IV pole transportation device that allows the user to easily connect multiple IV poles to facilitate the simultaneous transport of said IV poles. The IV pole transportation device comprises a base plate comprising a plurality of slots and a plurality of ratchet assemblies. The ratchet assemblies are secured to the base plate and positioned such that each ratchet assembly slot is aligned with a slot in the base plate.

The present invention is also directed to a method for using an IV pole transportation device. First, the method comprises providing an IV pole transportation device. The user then suspends the IV pole transportation device substantially parallel to the ground and positions a first IV pole in the ratchet assembly slot. The IV pole is secured by actuating the ratchet assembly, thereby compressing the IV pole between the ratchet assembly slot and the ratchet jaws. Additional IV poles are secured to the IV pole transportation device until all the available ratchet assemblies are in use. The user then wheels the entire configuration of the IV pole transportation device and the attached IV poles to the desired location.

### BRIEF DESCRIPTION OF THE DRAWINGS

These and other features, aspects and advantages of the present invention will become better understood with reference to the following description, appended claims, and accompanying drawings where:

FIG. 1 shows a top view of one embodiment of the present invention.

FIG. 2 shows a detail view of one element of the present invention.

FIG. 3 shows a top view of one embodiment of the present invention.

FIG. 4 shows a top view of one embodiment of the present invention.

FIG. 5 shows an exploded side view of an embodiment of the ratchet assembly of the present invention.

FIG. 6 shows a bottom view of an element of the present invention.

FIG. 7 shows a bottom view of an element of the present invention.

FIG. 8 shows a bottom view of an embodiment of the ratchet and ratchet receiving disc of the present invention.

FIG. 9 shows a top view of an element of the present invention.

FIG. 10 shows a top view of an element of the present invention.

FIG. 11 shows the steps of a method of the present invention.

### DETAILED DESCRIPTION OF THE INVENTION

The present device and method allow a user to transport multiple IV poles simultaneously. Specifically, the device of the present invention quickly, reversibly and securely engages a plurality of IV poles, forming an integrated unit that is stable and easy to transport.

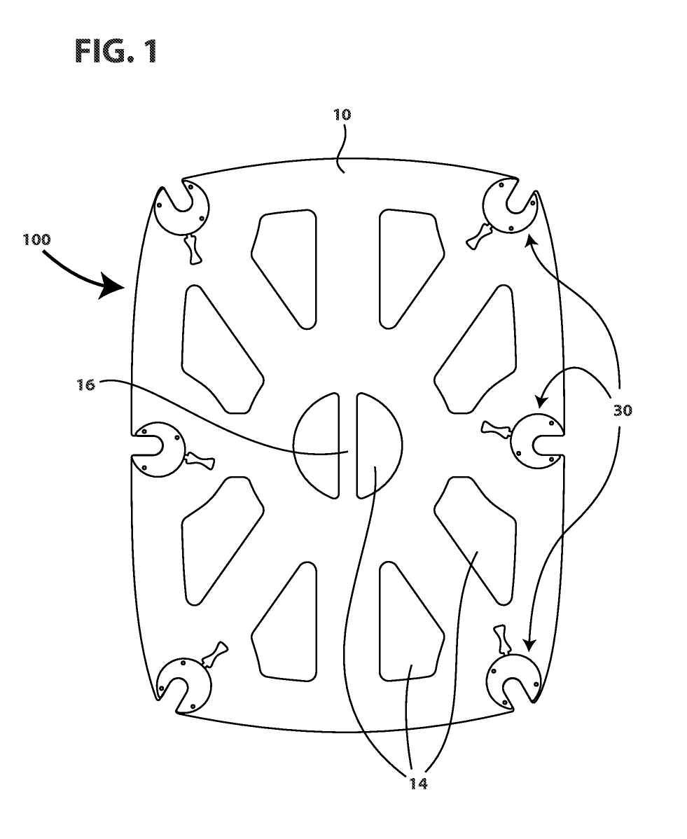

The present invention addresses the problems surrounding the simultaneous transportation of multiple IV poles. FIG. 1 shows one embodiment of the IV pole transportation device 100 of the present invention. The IV pole transportation device 100 comprises a base plate 10 and a plurality of ratchet assemblies 30 disposed about the perimeter of the base plate 10. The ratchet assemblies 30 are spaced to ensure that IV poles can be secured to the IV pole transportation device 100 without tangling the wheeled bases of the IV poles. Once secured to the IV pole transportation device 100, the wheeled bases of the IV poles allow the user to easily maneuver the IV pole transportation device 100 and the attached IV poles from one location to another. In addition, once the IV pole transportation device 100 secures a group of IV poles, the entire configuration in significantly more stable that an individual IV pole. As a result, the group of IV poles secured to the IV pole transportation device 100 may be transported without concern that the IV poles will tip over and any equipment attached to the IV poles may be transported without concern that it will be damaged in a fall. Additionally, once the IV pole transportation device 100 is secured to a group of IV poles, the IV pole transportation device 100 provides a stable flat surface that may be used to hold additional small items during transport.

Preferably, the base plate 10 is made of polycarbonate. However, the base plate 10 may be made from any suitable material know to persons of skill in the art to possess the requisite rigidity, durability and weight, including but not limited to wood, plastic, metal, composite or combinations thereof. Cutouts 14 are not a required element of the IV pole transportation device 100. However, cutouts 14 are preferred because they reduce the weight of the base plate 10 and provide locations where a user can easily grasp the IV pole transportation device 100. As shown in FIG. 1, the base plate 10 of the IV pole transportation device 100 includes a plurality of cutouts 14, including a pair of cutouts 14 that form a handle 16. Preferably, the handle 16 is located near the center of the base plate 10 so that a user can easily balance the IV pole transportation device 100 with one hand, while using the other hand to engage and secure IV poles. In other embodiments of the base plate 10, the specific size, shape and arrangement of the cutouts 14 can vary widely and is only limited by the desired rigidity and durability of the base plate 10.

In addition to the handle 16 and the hand holding positions created by the position of the cutouts 14, the IV pole transportation device 100 may incorporate one or more tow handles. Persons of skill in the art will recognize that tow handles are defined as handles which extend beyond the periphery of the base plate 10 and are used to maneuver the IV pole transportation device 100 during the process of transporting IV poles from one location to another.

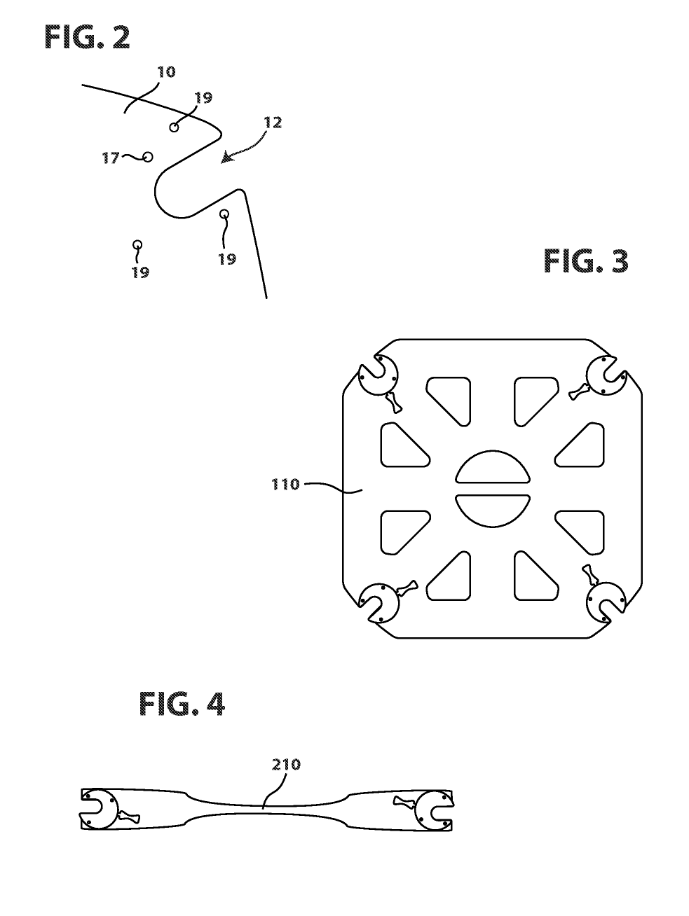

Turning to FIG. 2, a detail of the base plate 10 is shown. The base plate 10 includes a slot 12 at each location where a ratchet assembly 30 is installed. Each slot 12 is positioned at the edge of the base plate 10 and is sized and shaped to accept an IV pole. Surrounding the slot 12 is a series of assembly screw holes 19, which allow a screw or bolt to pass through the base plate 10 and secure the ratchet assembly 30. A spring hole 17, which is part of the ratchet locking mechanism, is also located near the slot 12 and extends through the full width of the base plate 10.

The number and arrangement of ratchet assemblies 30 dictates the maximum number of IV poles that can be transported with a device of the present invention. The IV pole transportation device 100 shown in FIG. 1 has six ratchet assemblies 30 and can secure a maximum of six IV poles. Preferably, the device of the present invention comprises between two and ten ratchet assemblies 30. FIGS. 3 and 4 show alternative embodiments of the IV pole transportation device of the present invention, with FIG. 3 depicting an IV pole transportation device 200 having a base plate 110 adapted to engage four IV poles and FIG. 4 depicting an IV pole transportation device 300 having a base plate 210 adapted to engage two IV poles. Persons of skill in the art will appreciate that in addition to these specific examples, numerous alternative configurations of the device of the present invention that allow for simultaneous transport of a ranging number of IV poles are possible.

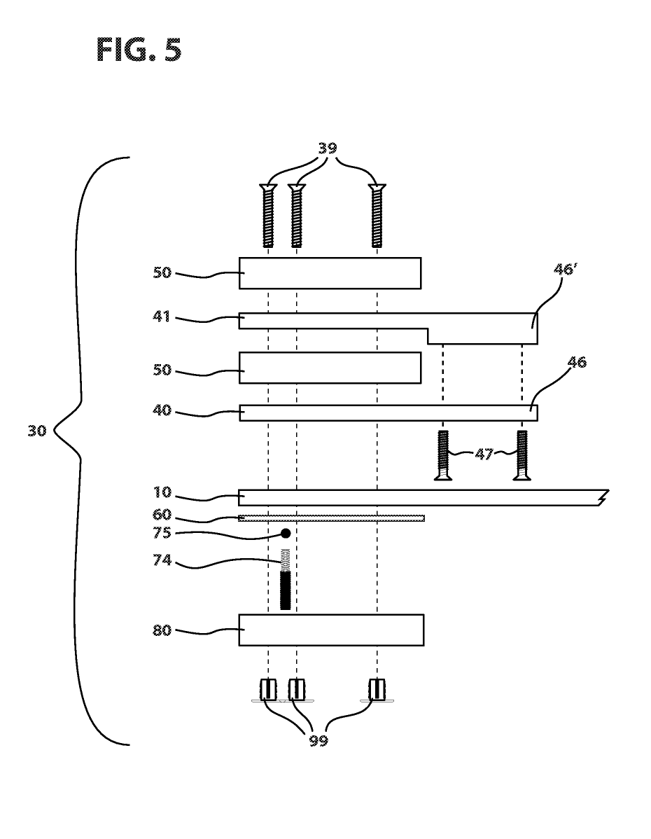

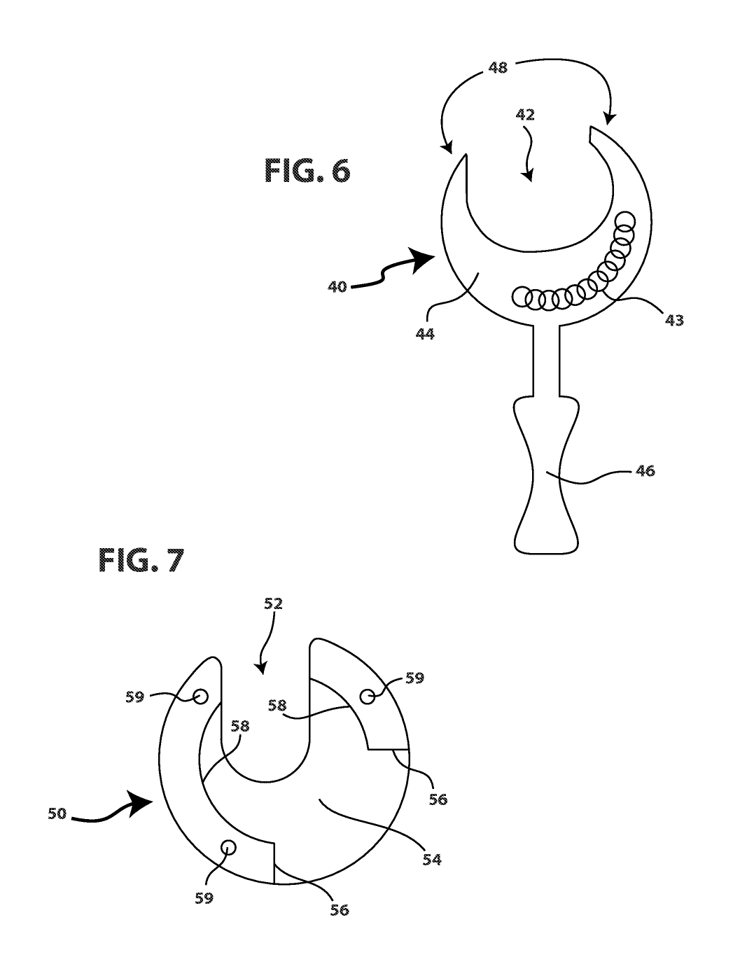

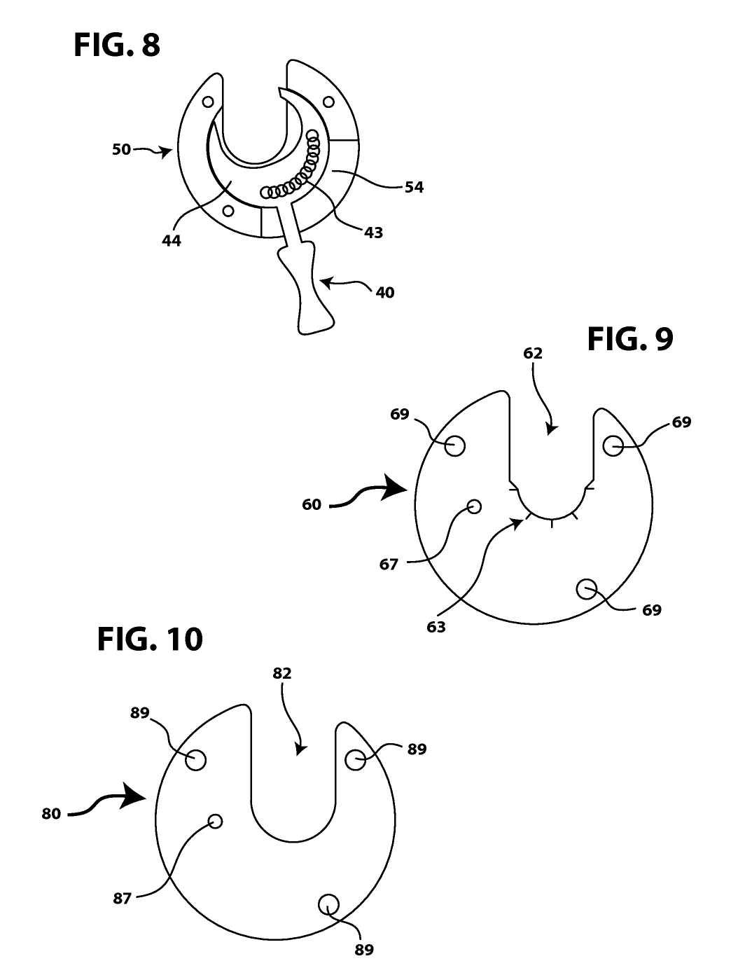

FIG. 5 shows the components of an embodiment of the ratchet assembly 30 of the present invention, while FIGS. 6-10 show additional details for each component of the ratchet assembly 30. The arrangement of ratchet assembly 30 components depicted in FIG. 5 are held together and secured to the base plate 10 using assembly screws 39, which pass through holes in each of the ratchet receiving discs 50, the base plate 10, the anti slip disc 60 and terminate in a corresponding threaded insert 99. The ratchet assembly 30 comprises at least a ratchet 40 and a ratchet receiving disc 50, but preferably includes an upper ratchet 41 and a second ratchet receiving disc 50 to increase the strength with which the ratchet assembly 30 engages the IV poles.

As depicted in FIG. 6, the ratchet 40 includes a handle 46. In addition, the ratchet 40 is molded or machined to include an oval slot 42, which is offset in a manner that creates an offset cam. The oval slot 42 defines a pair of ratchet jaws 48, which contact and secure the IV pole when the ratchet assembly is actuated. The underside of the ratchet 40 includes a ratcheting track 43, which is machined or molded to be dimpled and to interface with a ball bearing 75. During actuation of the ratchet assembly 30, the ball bearing 75 provides resistance and a locking action as ball bearing 75 is forced to move from one dimpled segment of the ratcheting track 43 to another. Preferably, the ratchet 40 is made of acetal, but persons of skill in the art will appreciate that suitable materials for the ratchet 40 include, but are not limited to wood, plastic, metal, composite or combinations thereof.

The present invention encompasses ratchet assemblies 30 that include a single ratchet 40 as well as ratchet assemblies 30 that include two or more ratchets 40. FIG. 5 depicts an embodiment of the ratchet assembly 30 comprising a ratchet 40 and an upper ratchet 41. While the upper ratchet 41 is preferably substantially identical to the ratchet 40 in shape and size, there may be difference in the ratchet 40 and the upper ratchet 41. For example, the upper ratchet 41 may omit the ratcheting track 43 or the upper ratchet 41 may be a different thickness that the ratchet 40 or the handle 46 may differ slightly.

Where a ratchet 40 and one or more upper ratchets 41 are utilized in a ratchet assembly 30, it is desirable to ensure that the ratchet 40 and the upper ratchet 41 will rotate in unison. Accordingly, when a ratchet 40 and an upper ratchet 41 are used in a ratchet assembly 30, the ratchet 40 and the upper ratchet 41 are preferably fixedly connected via their handles 46. One method, depicted in FIG. 5, to fixedly connect the handles 46 of the ratchet 40 and upper ratchet 41 is to use ratchet handle screws 47, which screw into predrilled holes in the ratchet handles 46. Alternatively, the ratchet handles 46 may be fixedly connected using pins or glue.

As described in more detail below, each ratchet 40 is mated with a ratchet receiving disc 50. Accordingly, when a ratchet 40 and an upper ratchet 41 are utilized in a ratchet assembly, a gap is created between the ratchet 40 handle 46 and the upper ratchet 41 handle 46 due to the inclusion of an additional ratchet receiving disc 50. While this gap is primarily cosmetic and may be maintained without decreasing the functionality of the ratchet assembly 30, it is preferable to modify the handle 46 of the ratchet 40, the handle 46 of the upper ratchet 41, or both, such that the handles 46 come in direct contact with each other. One alternative of an altered handle 46′ is shown in FIG. 5. Alternatively, a spacer may be used to bridge any gap between the handles 46.

Where it is desirable to increase the force and holding power of the ratchet assembly 30, but undesirable to utilize a ratchet 40 and one or more upper ratchets 41, the width of the ratchet 40 may be expanded to increase the surface area that comes in contact with the IV pole during operation.

As shown in FIG. 7, the ratchet receiving disc 50 includes a pocket 54 and a slot 52. The pocket 54 is machined or molded to be approximately the same depth as the width of the ratchet 40 and permits the nesting of the ratchet 40 inside the ratchet receiving disc 50. In addition, the side walls 58 of the pocket 54 substantially mirror the outer radius of the ratchet body 44 and secure the ratchet 40 in place within the ratchet assembly 30. The pocket 54 also includes two shoulders 56, which contact the ratchet handle 46 and limit the rotation of the ratchet 40 to approximately 90 degrees. The shoulder opposite the slot 52 corresponds to the open position of the ratchet assembly 30, and the shoulder substantially perpendicular to the slot 52 corresponds to the maximum closed position of the ratchet assembly 30. The slot 52 is machined or molded such that it is offset, which ensures that the slot 52 will not be obstructed by the ratchet jaws 48 when the ratchet assembly 30 is in the open position.

The height of the ratchet receiving disc 50, and especially the depth of the pocket 54, is dictated by the thickness of the ratchet 40. Specifically, the depth of the pocket 54 is approximately the same as the thickness of the ratchet 40 to ensure that the ratchet 40 is held securely in place, but is not held so tightly that the ratchet 40 cannot rotate. In embodiments where the ratchet assembly 30 includes a ratchet 40 and an upper ratchet 41 of differing thicknesses, the depth of the slot 54 in each ratchet receiving disc 50 will also vary, with the depth of each pocket 54 corresponding to the thickness of the ratchet 40 nested inside the ratchet receiving disc 50. Preferably, the ratchet receiving disc 50 is made of polycarbonate, but persons of skill in the art will appreciate that suitable materials for the ratchet receiving disc 50 include, but are not limited to wood, plastic, metal, composite or combinations thereof.

As shown in FIG. 5, the ratchet assembly may include an anti slip disc 60. Preferably, the anti slip disc 60 is made of vinyl or some other material with a larger coefficient of friction than the hard plastics that are preferred for the other components of the ratchet assembly 30. The anti slip disc includes a slot 62 that is approximately the same size and shape as the slot 12 in the base plate and is offset to align with the slot 52 in the ratchet receiving discs 50. At the base of the slot 62, there is a series of wings 63 that extend beyond the edge of the base plate 10 and into the slot 12 when the anti slip disc 60 is installed on the base plate 10. When an IV pole is inserted into the slot 12, the IV pole comes in contact with the wings 63. Further, as the ratchet assembly 30 is actuated, the IV pole is compressed against the wings 63 as the ratchet jaws 48 press against the IV pole. The resulting frictional forces increase the grip of the IV pole transportation device 100 on the IV pole and prevent the IV pole transportation device 100 from slipping down the IV pole even when small items are placed atop the IV pole transportation device 100.

The base disc 80 substantially mirrors the ratchet receiving disc 50 in shape, size and construction and includes threaded insert holes 89, sized to accept to accept the threaded inserts 99. Similar to the other components of the ratchet assembly, the base disc 80 includes a slot 12, which is approximately the size and shape as the slot 12 in the base plate 10 and is offset to align with slot 52 in the ratchet receiving discs 50. The base disc 80 also includes a spring hole 87, which is aligned with the spring hole 17 in the base plate 10 and the spring hole 67 in the anti slip disc 60. However unlike the other spring holes 17, 67, which are through holes, the base disc 80 spring hole 87 is a blind hole that does not extend the full width of the base disc 80. The spring hole 87 is sized to accept a spring 74, which is inserted into the spring hole 87 and supported by the spring hole 87 bottom.

The spring 74 supports a ball bearing 75, which is sized and shaped to interface with the ratcheting track 43. The bottom of the spring hole 87, the spring 74, the ball bearing 75 and the ratcheting track 43 comprise the locking mechanism of the ratchet assembly 30. The resistance and holding power of the locking mechanism can be adjusted based on the tension of the spring 74, whereby the more force the spring 74 exerts on the ball bearing 75 once the ratchet assembly 30 is constructed, the tighter the locking mechanism will be.

To construct the portion of the ratchet assembly 30 located above the base plate, the upper ratchet 41 is fitted into the pocket 54 of a first ratchet receiving disc 50 and the first ratchet receiving disc 50 is stacked on top of a second ratchet receiving disc 50 such that the upper ratchet 41 is sandwiched between the two ratchet receiving discs 50. Similarly, the ratchet 40 is inserted into the pocket 54 of the second ratchet receiving disc 50 and the second ratchet receiving disc 50 is stacked on top of the base plate 10 such that the ratchet 40 is sandwiched between the second ratchet receiving disc 50 and the base plate 10. The two ratchet receiving discs are aligned with the base plate 10 such that the slots 12, 52 and assembly screw holes 19, 59 are aligned.

To construct the portion of the ratchet assembly 30 positioned below the base plate 10, the anti slip disc 60 is stacked on top of the base disc 80, such that the assembly screw holes 69 are aligned with the threaded inserts 99 that are positioned in the threaded insert holes 89. In addition, the spring holes 67, 87 are aligned. The spring is inserted through the spring hole 67, into the spring hole 87 and rests against the bottom of the spring hole 87. A ball bearing 75 is positioned atop the free end of the spring 74 and the base disc 80 and anti slip disc 60 are stacked against the base plate 10 opposite the ratchet receiving discs 50. As the base disc 80 and anti slip disc 60 are maneuvered into position, the bearing 75 and spring 74 pass through the spring hole 17 such that the bearing contacts the ratcheting track 43 in the underside of the ratchet 40 and the spring 74 and ball bearing 75 are sandwiched between the bottom of the spring hole 87 and the ratchet 40.

When properly configured, the slots 12, 52, 62, 82 are aligned with each other to form a ratchet assembly 30 slot, and the assembly holes 19, 59, 69 and are aligned with each other and the threaded inserts 99. To secure the ratchet assembly 30, the assembly screws 39 are inserted through the assembly screw holes 59, 19, 69 and secured into the threaded inserts 99. Once constructed, the ratchet assembly 30 may be actuated by moving the handles 46 from the open to the closed position. Preferably, the handles 46 are fixedly secured to one another. However, in some embodiments the handles 46 are not attached.

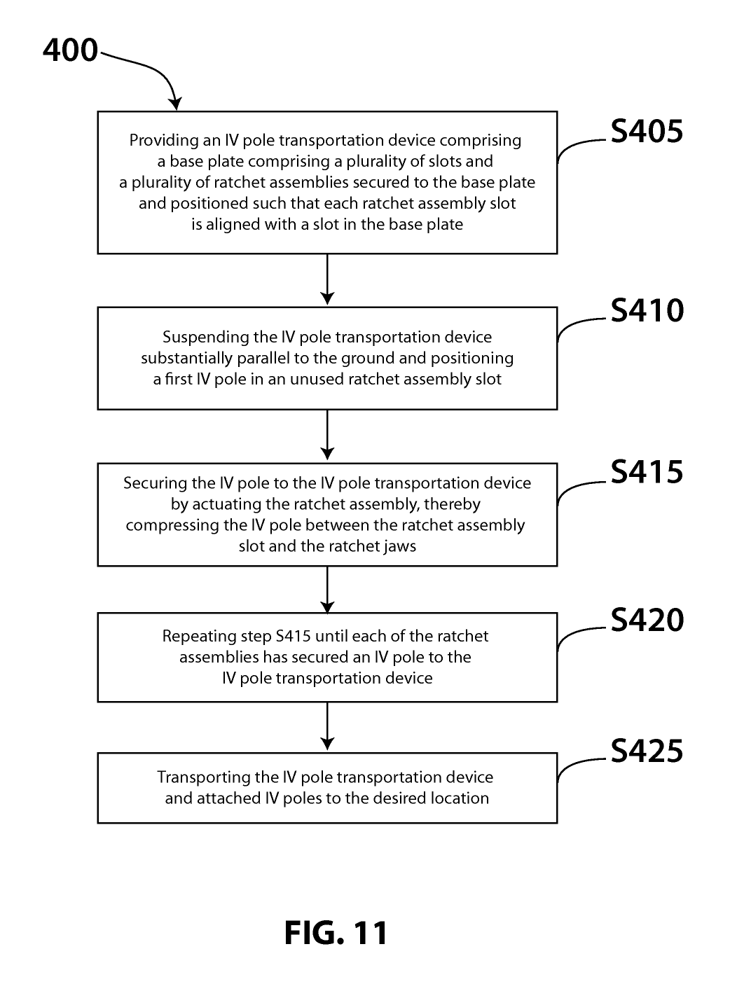

Turning now to FIG. 11, one embodiment of the method 400 of the present invention is depicted. A first step S405 comprises providing a IV pole transportation device 100 and a second step S410 comprises suspending the IV pole transportation device 100 substantially parallel to the ground and positioning a first IV pole in the slot 12 of the base plate 10. A third step S415 comprises securing the IV pole to the IV pole transportation device 100 by moving the handles 46 from the open to the closed position, thereby actuating the ratcheting assembly 30 and rotating the ratchet 40 and the upper ratchet 41. As the ratchet 40 and upper ratchet 41 are rotated, the ratchet jaws 48 contact the IV pole and secure it against the wings 63 of the anti slip disc 60 and the inner edge of the slots 12, 52, 82. The amount of rotation required to secure a given IV pole to the IV pole transportation device 100 will depend on the diameter of the IV pole and it is preferable that the oval cutout 42 in sized such that most standard IV poles can be secured before the ratchet 40 and upper ratchet 41 achieve their maximum degree of rotation.

A fourth step S420 comprises repeating step S415 until each of the ratchet assemblies 30 has secured an IV pole to the IV pole transportation device 100. Alternatively, in situations where the number of IV poles being transported is less than the number of ratchet assemblies 30, step S420 would be repeated until each of the IV poles were attached, leaving empty ratchet assemblies 30. Once a second IV pole is secured to the IV pole transportation device 100, the user may release the handle 16 and the IV pole transportation device 100 will remain suspended and securely attached to the IV poles, freeing the user to move about to grab additional IV poles.

As explained above, once the IV pole transportation device 100 is secured to a group of IV poles, the entire configuration may be wheeled from one location to another using the wheeled bases of the attached IV poles. Therefore, a fifth step S425 comprises grasping the IV pole transportation device 100 and transporting the device and attached IV poles to the desired location by pushing or pulling the IV pole transportation device 100.

The present method is significantly more efficient than the current alternatives of individually transporting IV poles. In addition, by securing the IV poles to the IV pole transportation device 100 the entire configuration is significantly more stable than an individual IV pole, which reduces the risk that the IV poles will tip over during transport and makes it safer to transport IV poles and any attached medical equipment. An additional benefit of the present invention is that once the IV pole transportation device 100 is secured to a group of IV poles, the IV pole transportation device 100 itself may function as a cart by holding small items during transport.

It is noted that the foregoing examples have been provided merely for the purpose of explanation and are in no way to be construed as limiting of the present invention. While the present invention has been described with reference to exemplary embodiments, it is understood that the words, which have been used herein, are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

1. An IV pole transportation device comprising:
a base plate comprising a plurality of slots; and
a plurality of ratchet assemblies secured to the base plate and positioned such that each ratchet assembly slot is aligned with a base plate slot.

2. The IV pole transportation device of claim 1 further comprising a cutout in the base plate.

3. The IV pole transportation device of claim 2 further comprising a handle in the base plate.

4. The IV pole transportation device of claim 1 wherein each ratchet assembly includes one ratchet.

5. The IV pole transportation device of claim 1 wherein each ratchet assembly includes a plurality of ratchets.

6. The IV Pole transportation device of claim 4 wherein the ratchet is constructed from a material selected from the group consisting of plastic, metal, composite, wood or combinations thereof.

7. The IV Pole transportation device of claim 6 wherein the ratchet is constructed from acetal.

8. The IV pole transportation device of claim 1 wherein each ratchet assembly includes an anti slip disc.

9. The IV pole transportation device of claim 5 wherein the plurality of ratchets each include a handle and the handles of the plurality of ratchets are fixedly attached to each other.

10. The IV Pole transportation device of claim 1 wherein the base plate is constructed of a material selected from the group consisting of plastic, metal, composite, wood or combinations thereof.

11. The IV Pole transportation device of claim 10 wherein the base plate is constructed of polycarbonate.

12. An IV pole transportation device comprising:
a. a base plate comprising a plurality of slots;
b. a plurality of ratchet assemblies secured to the base plate wherein each ratchet assembly comprises:
i. a lower ratchet receiving disc having a slot and mounted on top of the base plate such that the lower ratchet receiving disc slot is aligned with one of the base plate slots;
ii. a lower ratchet nested inside the lower ratchet receiving disc such that lower ratchet is sandwiched between the base plate and the lower ratchet receiving disc;
iii. an upper ratchet receiving disc having a slot and mounted on the lower ratchet receiving disc such that the upper ratchet receiving disc slot is aligned with the lower ratchet receiving disc slot;
iv. an upper ratchet nested inside the upper ratchet receiving disc such that upper ratchet is sandwiched between the lower ratchet receiving disc and the upper ratchet receiving disc;
v. a base disc having a slot and mounted below the base plate such that the base disc slot is aligned with the base plate slot, the lower ratchet receiving disc slot and the upper ratchet receiving disc slot.

13. The IV pole transportation device of claim 12 wherein the ratchet assemblies further comprise an anti slip disc having a slot and mounted below the base plate such that the anti slip disc is sandwiched between the base disc and the base plate and the anti slip disc slot is aligned with the base disc slot, the base plate slot, the lower ratchet receiving disc slot and the upper ratchet receiving disc slot.

14. The IV pole transportation device of claim 12 further comprising a cutout in the base plate.

15. The IV pole transportation device of claim 12 further comprising a handle in the base plate.

16. The IV pole transportation device of claim 12 wherein the upper ratchet and lower ratchet are constructed of a material selected from the group consisting of plastic, metal, composite, wood or combinations thereof.

17. The IV pole transportation device of claim 16 wherein the upper ratchet and lower ratchet are constructed of acetal.

18. A method for transporting IV pole comprising:
a. providing an IV pole transportation device comprising:
i. a base plate comprising a plurality of slots; and
ii. a plurality of ratchet assemblies secured to the base plate and positioned such that each ratchet assembly slot is aligned with a base plate slot;
b. suspending the IV pole transportation device substantially parallel to the ground and positioning a first IV pole in an unused ratchet assembly slot;
c. securing the IV pole to the IV pole transportation device by actuating the ratchet assembly, thereby compressing the IV pole between the ratchet assembly slot and the ratchet jaws;
d. repeating step c until each of the ratchet assemblies has secured an IV pole to the IV pole transportation device;
e. transporting the IV pole transportation device and attached IV poles to the desired location.

19. The method of claim 18 wherein each ratchet assembly includes an anti slip disc.
